(19) United States
(12) Patent Application Publication
(10) Pub. No.: US 2026/0306577 A1
(43) Pub. Date: Oct. 8, 2026

(54) PRODUCTION METHOD

(71) Applicant: AG IP COMPANY PTY LTD, lraak, Victoria (AU)

(72) Inventor: Nicholas DIAMANTOPOULOS, Iraak, Victoria (AU)

(21) Appl. No.: 19/481,418

(22) PCT Filed: May 3, 2024
(86) PCT No.: PCT/AU2024/050431

§ 371 Date: Nov. 4, 2025

(30) Foreign Application Priority Data
May 4, 2023 (AU) 2023901346

(51) Int. Cl. A61K 36/8962; A61P 31/14; A61P 31/16; B01D 3/10; B01D 11/02; C11B 1/04; C11B 1/10

(52) U.S. Cl. CPC A61K 36/8962; A61P 31/14; A61P 31/16; B01D 3/10; B01D 11/0203; C11B 1/04; C11B 1/104; A61K 2236/11; A61K 2236/15; A61K 2236/37

(57) ABSTRACT

The invention relates, inter alia, to methods of preparing a garlic oil, garlic oils produced using the method, pharmaceutical compositions comprising the garlic oil and uses of the garlic oil in the treatment of viral infections such as SARS-COV-2 and influenza A. The methods of preparing a garlic oil comprise the steps of: a) processing a portion of a garlic plant to increase its surface area; b) ageing the portion of the garlic plant for at least 18 months to provide an aged garlic, which does not comprise addition of ethanol; and c) separating the garlic oil in the aged garlic. Step c) may be performed using spinning cone distillation or supercritical fluid extraction, at a temperature below 110° C., or such that the pressure (atmospheres) divided by the temperature (degrees Kelvin) is equal to or less than about 0.0026.

### TECHNICAL FIELD

In one embodiment the present invention relates to methods of producing a garlic oil. In other embodiments, the invention relates to garlic oil produced by the method, and to methods and uses involving garlic oil.

### BACKGROUND ART

It will be clearly understood that, if a prior art publication is referred to herein, this reference does not constitute an admission that the publication forms part of the common general knowledge in the art in Australia or in any other country.

Garlic is known to contain volatile organic compounds exhibiting a wide array of biological effects, such as antioxidant, immunomodulatory, anti-fungal, anti-inflammatory, anti-bacterial and anti-viral activity. Organosulfates are a type of volatile organic compound in garlic which are often attributed to have these beneficial effects. As a result of its broad activity, garlic preparations are commonly used as a dietary supplement and in herbal medicine, marketed as being effective against a variety of conditions.

Fresh garlic can be used but suffers from a short shelf life and must be consumed raw for maximum efficacy, as cooking the garlic generally causes degradation of the bioactive compounds therein. Additionally, fresh garlic only contains about 2-4% by weight organosulfates, and can have a strong taste and odour which can be unpalatable to consumers. In order to overcome some of these difficulties, a range of techniques have been used to extract the bioactive compounds from garlic.

One way to produce a garlic preparation is through maceration. In this method, chopped garlic is steeped in a liquid such as water, ethanol, or a carrier oil such as soybean or vegetable oil for several days to up to several weeks. This causes active compounds in the raw garlic to be extracted into the liquid, which is then filtered to remove any remaining raw garlic. While maceration does avoid the use of high temperatures which may degrade the bioactive compounds, the resulting concentration of active components is necessarily low due to the large excess of liquid and thus the biological activity is substantially reduced. Additionally, such preparations often create emulsions or liquids with a high-water content, which can be microbially unstable and thus have a reduced shelf life.

In order to produce a more microbially stable product, some methods form a garlic oil which is substantially free of water. One such method is through chemical extraction. In this method, garlic is washed with a solvent in order to extract the active components into the solvent. The solvent is then separated from the garlic, filtered and concentrated to remove the solvent and any excess moisture. This generally results in an oily composition containing compounds obtained from the garlic. While these compositions have greater microbial stability compared to aqueous formulations, many of the bioactive compounds present in garlic are volatile, and are thus lost when the solution is concentrated.

Commercial garlic oil is generally produced using distillation techniques such as steam distillation. During steam distillation, fresh garlic is mixed with water and the mixture is heated until the water boils. The steam produced from the boiling water then carries the volatile compounds to a cooling system, which condenses the volatile-rich steam into a liquid. The liquid then spontaneously separates into an aqueous phase and an oil phase, allowing collection of the pure garlic oil via decantation. While steam distillation allows for obtaining a garlic oil comprising volatile compounds, the solution must be boiled for a prolonged period of time to obtain sufficient yield. This prolonged exposure to heat can cause degradation of the bioactive compounds within garlic, resulting in a substantial loss in biological activity of the resultant oil.

Accordingly, the concentration of bioactive compounds in garlic preparations can vary widely, and many methods produce a composition that is either not shelf stable, has a low bioactive concentration, or has caused degradation of the key bioactive compounds present in garlic.

### SUMMARY OF INVENTION

With the foregoing in view, the present invention in one aspect relates to a method of producing garlic oil which retains or improves the biological activity of the garlic.

In a first aspect, the present invention provides a method of preparing a garlic oil, the method comprising the steps of:
a) processing a portion of a garlic plant to increase its surface area;
b) ageing the portion of the garlic plant for at least 18 months to provide an aged garlic; and
c) separating the garlic oil in the aged garlic, wherein the separating is performed using spinning cone distillation or supercritical fluid extraction.

In an embodiment of the first aspect, there is provided a method of preparing a garlic oil, the method comprising the steps of:
a) processing a portion of a garlic plant to increase its surface area;
b) ageing the portion of the garlic plant for at least 18 months to provide an aged garlic; and
c) separating the garlic oil in the aged garlic, wherein the separating is performed using spinning cone distillation or supercritical fluid extraction,
wherein step b) does not comprise addition of ethanol.

In a second aspect of the invention, there is provided a method of preparing a garlic oil, the method comprising the steps of:
a) processing a portion of a garlic plant to increase its surface area;
b) ageing the portion of the garlic plant for at least 18 months to provide an aged garlic; and
c) separating the garlic oil in the aged garlic, wherein the separating is performed at a temperature below 110° C.

In an embodiment of the second aspect, there is provided a method of preparing a garlic oil, the method comprising the steps of:
a) processing a portion of a garlic plant to increase its surface area;
b) ageing the portion of the garlic plant for at least 18 months to provide an aged garlic; and
c) separating the garlic oil in the aged garlic, wherein the separating is performed at a temperature below 110° C.,
wherein step b) does not comprise addition of ethanol.

In a third aspect of the invention, there is provided a method of preparing a garlic oil, the method comprising the steps of:
a) processing a portion of a garlic plant to increase its surface area;
b) ageing the portion of the garlic plant for at least 18 months to provide an aged garlic; and
c) separating the garlic oil in the aged garlic, wherein during the separating the pressure (atmospheres) divided by the temperature (degrees Kelvin) is equal to or less than about 0.0026.

In an embodiment of the third aspect, there is provided a method of preparing a garlic oil, the method comprising the steps of:
a) processing a portion of a garlic plant to increase its surface area;
b) ageing the portion of the garlic plant for at least 18 months to provide an aged garlic; and
c) separating the garlic oil in the aged garlic, wherein during the separating the pressure (atmospheres) divided by the temperature (degrees Kelvin) is equal to or less than about 0.0026,
wherein step b) does not comprise addition of ethanol.

Features of the first to third aspects (and the embodiments of the first to third aspects described above) may be as described below.

Advantageously, the present method provides a garlic oil which substantially retains or improves the biological activity of the garlic.

Without wishing to be bound by any theory, the inventors believe that the ageing process advantageously causes breakdown in the cellular structures within the garlic, thereby releasing the oils and active compounds contained within the intracellular environment. Furthermore, the ageing process converts unstable, reactive compounds into more stable analogs.

As used in the present specification, the term “garlic oil” refers to a hydrophobic solution derived from a part of a garlic plant. While the garlic oil may include other types of oils (for example a mixture with an oil derived from another plant), in some embodiments the garlic oil consists of (or consists essentially of) only oil derived from a garlic plant. The term “garlic oil” as used herein may also comprise a substantially small water content, but the oil as a whole is hydrophobic and may not be an emulsion.

The portion of a garlic plant in step a) may be whole garlic bulbs, unpeeled garlic cloves or peeled garlic cloves, or a combination thereof.

In some embodiments, in step a) the portion of a garlic plant is peeled garlic cloves.

In some embodiments, the portion of a garlic plant is step a) is from or is derived from an Australian garlic variety; especially Australian Red (MOF), Australian White (WHT), or Australian Purple (AUP) garlic variety.

As used herein, the term “Australian Red (MOF)” refers to a hard neck garlic variety, with 8 to 12 pink, red or purple garlic cloves and the bulb has white external skins. This variety is frequently referred to as Morado in Europe, Creole & Rojo in USA and Colorado in Argentina. In Australia, this garlic variety would be typically harvested in about December.

As used herein, the term “Australian White (WHT)” refers to a soft neck garlic variety, with 10 to 16 white, yellow, or light brown garlic cloves and the bulb has white external skins. This variety is often referred to as Spanish White, Italian White, Messidrome or Thermidrome in Europe, Artichoke or early white in the USA and Blanco in Argentina. In Australia, this garlic variety would be typically harvested in about November.

As used herein, the term “Australian Purple (AUP)” refers to a hard neck garlic variety, with 8 to 14 brown, pink, red or purple garlic cloves and the bulb has light purple external skins. This variety is often referred to as Spring Purple in Europe, Turban or Chinese Purple in USA and Chino/Asiatico Morado in Argentina. In Australia, this variety would be typically harvested in about October.

In some embodiments, the portion of a garlic plant is not from, or is not derived from, a Griffith White garlic variety.

As used herein, the term “Griffith White (AUW)” refers to a hard neck garlic variety, with 8 to 14 yellow, brown or light pink garlic cloves and the bulb has white or very faint purple thin external skins. This variety is often referred to as Sprint, Primor or Spring White in Europe, Turban or Chinese White in USA and Chino Morado Blanco in Argentina. In Australia, this variety would be typically harvested in about October.

In some embodiments, step a) comprises mincing, cutting, chopping, dicing, grinding, crushing, comminuting, triturating, blending, threshing, pulverising, mashing, squashing, cubing, dividing, milling, powdering, granulating, smashing, grating, pounding, pressing, thrashing or any combination thereof.

Step a) may comprise cutting, grinding, crushing or any combination thereof. In one embodiment, cutting comprises chopping, dicing, cubing, dividing, blending, comminuting, grating or any combination thereof. In another embodiment, crushing comprises grinding, thrashing, threshing, pulverising, mashing, squashing, smashing, pounding, pressing or any combination thereof. In a further embodiment griding comprises triturating, milling, powdering, granulating or any combination thereof.

In some embodiments, step a) comprises mincing.

In some embodiments, step a) reduces the size of the portion of a garlic plant to about 0.1 mm, 0.2 mm, 0.3 mm, 0.4 mm, 0.5 mm, 0.6 mm, 0.7 mm, 0.8 mm, 0.9 mm, 1.0 mm, 1.25 mm, 1.5 mm, 1.75 mm, 2.0 mm, 2.25 mm, 2.5 mm, 2.75 mm, 3.0 mm, 3.25 mm, 3.5 mm, 3.75 mm, 4.0 mm, 4.25 mm, 4.5 mm, 4.75 mm, 5.0 mm, 5.5 mm, 6.0 mm, 6.5 mm or about 7.0 mm.

In other embodiments, step a) reduces the size of the portion of a garlic plant to less than 0.2 mm, less than 0.3 mm, less than 0.4 mm, less than 0.5 mm, less than 0.6 mm, less than 0.7 mm, less than 0.8 mm, less than 0.9 mm, less than 1.0 mm, less than 1.25 mm, less than 1.5 mm, less than 1.75 mm, less than 2.0 mm, less than 2.25 mm, less than 2.5 mm, less than 2.75 mm, less than 3.0 mm, less than 3.25 mm, less than 3.5 mm, less than 3.75 mm, less than 4.0 mm, less than 4.25 mm, less than 4.5 mm, less than 4.75 mm, less than 5.0 mm, less than 5.5 mm, less than 6.0 mm, less than 6.5 mm or less than 7.0 mm.

In further embodiments, the size of the portion of a garlic plant after step a) is greater than 0.1 mm, greater than 0.2 mm, greater than 0.3 mm, greater than 0.4 mm, greater than 0.5 mm, greater than 0.6 mm, greater than 0.7 mm, greater than 0.8 mm, greater than 0.9 mm, greater than 1.0 mm, greater than 1.25 mm, greater than 1.5 mm, greater than 1.75 mm, greater than 2.0 mm, greater than 2.25 mm, greater than 2.5 mm, greater than 2.75 mm, greater than 3.0 mm, greater than 3.25 mm, greater than 3.5 mm, greater than 3.75 mm, greater than 4.0 mm, greater than 4.25 mm, greater than 4.5 mm, greater than 4.75 mm, greater than 5.0 mm, greater than 5.5 mm, greater than 6.0 mm, greater than 6.5 mm or about 7.0 mm.

In one embodiment, step a) is performed without addition of solvent.

Step a) may be performed with the addition of solvent. In some embodiments the solvent is a polar solvent such as water, methanol, ethanol, isopropyl alcohol, ethyl acetate or combinations thereof. In other embodiments the solvent is a non-polar solvent such as hexane, plant based oils such as olive oil, canola oil, vegetable oil, sunflower seed oil and grapeseed oil or combinations thereof. In one embodiment, the polar solvent is not (or does not comprise) ethanol. In another embodiment, the polar solvent is not (or does not comprise) an alcohol.

In one embodiment, step a) is performed using only the portion of the garlic plant.

Step a) may be performed at any suitable pressure and temperature. In one embodiment, step a) is performed at atmospheric pressure. In another embodiment, step a) is performed below atmospheric pressure. In some embodiments, step a) is performed above atmospheric pressure. In another embodiment, step a) is performed at room temperature. In one embodiment, step a) is performed above room temperature. In another embodiment, step a) is performed below room temperature. In one embodiment, step a) is performed at −10° C., −5° C., 0° C., 5° C., 10° C., 15° C., 20° C., 25° C., 30° C., 35° C., 40° C., 45° C. or 50° C., or any temperature in between. In one embodiment, step a) is performed above 50° C., above 40° C., above 30° C., above 20° C., above 10° C., above 0° C. or above −10° C. In another embodiment, step a) is performed below 50° C., below 40° C., below 30° C., below 20° C., below 10° C., below 0° C. or below −10° C. In a further embodiment, step a) is performed at a temperature of from 0° C. to 50° C., especially from 10° C. to 40° C., or from 15° C. to 35° C.

In some embodiments, before step a), the method of producing garlic oil includes the step of harvesting garlic. The method of the first aspect may also include, before step a), the steps of separating green tops from garlic bulbs, cracking garlic bulbs and/or peeling garlic cloves.

In some embodiments, step b) does not comprise the addition of solvent. In other embodiments, step b) does not comprise addition of an aqueous solution, such as water. In further embodiments, step b) does not comprise addition of an alcohol, such as ethanol.

Without wishing to be bound by any theory, the inventors believe that addition of ethanol during the ageing step may have a deleterious effect on the aged garlic and hence the resultant garlic oil. This is because the ethanol has the potential to disrupt or damage the enzymes that are active during the ageing step, damage plant cells, and/or react with the active components in the garlic, causing unwanted chemical transformations or may inhibit the ageing process. There may also be some resistance amongst consumers associated with using solvents like ethanol to extract a plant based health product. For these reasons, addition of ethanol in step b) would ultimately result in a different profile of chemical compounds, which then is likely to have a very significant impact on the garlic oil separated in step c), which may not have the surprising activity exhibited by garlic oils of the present invention. Additionally, adding ethanol during step b) may impede step c), because any ethanol that is present in the solution may be separated alongside the garlic oil which may in turn impact on the recovery of volatile compounds. This may dilute the concentration of garlic oil in the final product significantly and cause further, more laborious purification steps to be required to separate the garlic oil from the ethanol.

Step b) may be performed at −20° C., −19° C., −18° C., −17° C., −16° C., −15° C., −14° C., −13° C., −12° C., −11° C., −10° C., −9° C., −8° C., −7° C., −6° C., −5° C., −4° C., −3° C., −2° C., −1° C., 0° C., 1° C., 2° C., 3° C., 4° C. or 5° C., or any temperature in between. In one embodiment, step b) is performed above −20° C., above −19° C., above −18° C., above −17° C., above −16° C., above −15° C., above −14° C., above −13° C., above −12° C., above −11° C., above −10° C., above −9° C., above −8° C., above −7° C., above −6° C., above −5° C., above −4° C., above −3° C., above −2° C., above −1° C., above 0° C., above 1° C., above 2° C., above 3° C. or above 4° C. In another embodiment, step b) is performed at a temperature below −19° C., below −18° C., below −17° C., below −16° C., below-15° C., below −14° C., below −13° C., below −12° C., below −11° C., below −10° C., below −9° C., below −8° C., below −7° C., below −6° C., below −5° C., below −4° C., below −3° C., below −2° C., below −1° C., below 0° C., below 1° C., below 2° C., below 3° C. or below 4° C. or below 5° C. in further embodiments, step b) is performed at a temperature from about −20° C. to about 5° C., about −20° C. to about 4° C., about −20° C. to about 3° C., about −20° C. to about 2° C., about −20° C. to about 1° C., about −20° C. to about 0° C., about −20° C. to about −1° C., about −20° C. to about −2° C., about −20° C. to about −3° C., about −20° C. to about −4° C., about −20° C. to about −5° C., about −20° C. to about −6° C., about −20° C. to about −7° C., about −20° C. to about −8° C., about −20° C. to about −9° C., about −20° C. to about −10° C., about −20° C. to about −11° C., about −20° C. to about −12° C., about −20° C. to about −13° C., about −20° C. to about −14° C., about −20° C. to about −15° C., about −20° C. to about −16° C., about −20° C. to about −17° C., about −20° C. to about −18° C., about −20° C. to about −19° C., about −20° C. to about 5° C., about −19° C. to about 5° C., about −18° C. to about 5° C., about −17° C. to about 5° C., about −16° C. to about 5° C., about −15° C. to about 5° C., about −14° C. to about 5° C., about −13° C. to about 5° C., about −12° C. to about 5° C., about −11° C. to about 5° C., about −10° C. to about 5° C., about −9° C. to about 5° C., about −8° C. to about 5° C., about −7° C. to about 5° C., about −6° C. to about 5° C., about −5° C. to about 5° C., about −4° C. to about 4° C., about −3° C. to about 3° C., about −2° C. to about 2° C., about −1° C. to about 1° C., about −5° C. to about 4° C., about −5° C. to about 3° C., about −5° C. to about 2° C., about −5° C. to about 1° C. or about −5° C. to about 0° C., 0° C. to about 1° C., 0° C. to about 2° C., 0° C. to about 3° C., 0° C. to about 4° C., 0° C. to about 5° C. or about −20° C. to about 0° C.

Step b) may be performed at a temperature of from about −3° C. to about 3° C.

In some embodiments, step b) does not comprise freezing. In other embodiments, step b) comprises freezing. In one embodiment, the freezing is lyophilization or freeze drying. Step b) may comprise thermal cycling the portion of a garlic plant, wherein one cycle comprises cooling the portion of the garlic plant until it is frozen and then warming the portion of the garlic plant until it is not frozen. In some embodiments, step b) comprises thermal cycling the portion of the garlic plant 1, 2, 3, 4, 5, 6, 7, 8, 9, 10, 11, 12, 13, 14, 15, 16, 17, 18, 19 or 20 times. In some embodiments, the portion of the garlic plant is thermal cycled more than 1, 2, 3, 4, 5, 6, 7, 8, 9, 10, 11, 12, 13, 14, 15, 16, 17, 18, 19 or 20 times. In other embodiments, the portion of the garlic plant is thermal cycled less than 2, 3, 4, 5, 6, 7, 8, 9, 10, 11, 12, 13, 14, 15, 16, 17, 18, 19 or 20 times.

In some embodiments, step b) is performed for about 1.5, 2, 3, 4, 5, 6, 7, 8, 9 or about 10 years, or any amount of time in between. In some embodiments, step b) is performed for at least 1.5 years, at least 2 years, at least 3 years, at least 4 years, at least 5 years, at least 6 years, at least 7 years, at least 8 years, at least 9 years or at least 10 years. In other embodiments, step b) is performed for less than 10 years, less than 9 years, less than 8 years, less than 7 years, less than 6 years, less than 5 years, less than 4 years, less than 3 years or less than 2 years. In another embodiment, step b) is performed for from 1.5 to 10 years, from 1.5 to 9 years, from 1.5 to 8 years, from 2 to 7 years, from 2 to 6 years, from 2 to 5 years, from 2 to 4 years, from 2 to 3 years, from 3 to 10 years, from 3 to 9 years, from 3 to 8 years, from 3 to 7 years, from 3 to 6 years, from 3 to 5 years or from 3 to 4 years. In some embodiments, step b) is performed for 18, 19, 20, 21, 22, 23 or 24 months.

Step b) may be performed for from about 3 to about 5 years.

In some embodiments, step b) is performed at atmospheric pressure. Step b) may be performed at a pressure below atmospheric pressure. Step b) may be performed at a pressure above atmospheric pressure.

In one embodiment, step b) is performed under an inert atmosphere. The inert atmosphere may be nitrogen (for example).

Step c) may be performed by spinning cone column distillation.

In some embodiments, step c) comprises adding an aqueous solution to the aged garlic to form a slurry before performing the spinning conc column distillation on the slurry. In one embodiment, the aqueous solution is water. In another embodiment, the aqueous solution is a mixture of water a solvent. In further embodiments, the aqueous solution is a mixture of water and an alcohol, such as ethanol. In some embodiments, the aqueous solution does not comprise an alcohol, or ethanol.

The slurry may be heated to about 50° C. before performing the spinning cone column distillation on the slurry.

The slurry may be heated to about 5° C., 10° C., 15° C., 20° C., 25° C., 30° C., 35° C., 40° C., 45° C., 50° C., 55° C., 60° C., 65° C., 70° C., 75° C., 80° C., 85° C., 90° C., 95° C. or about 99° C. or any temperature in between before performing the spinning cone column distillation on the slurry. In some embodiments, the slurry is heated to at least 5° C., 10° C., 15° C., 20° C., 25° C., 30° C., 35° C., 40° C., 45° C., 50° C., 55° C., 60° C., 65° C., 70° C., 75° C., 80° C., 85° C., 90° C. or 95° C. before performing the spinning cone column distillation on the slurry. In other embodiments, the slurry is heated to no more than 5° C., 10° C., 15° C., 20° C., 25° C., 30° C., 35° C., 40° C., 45° C., 50° C., 55° C., 60° C., 65° C., 70° C., 75° C., 80° C., 85° C., 90° C. or 95° C. before performing the spinning cone column distillation on the slurry. In some embodiments, the slurry is heated to from 5° C. to 95° C., from 10° C. to 90° C., from 15° C. to 85° C., from 20° C. to 80° C., from 25° C. to 75° C., from 30° C. to 70° C., from 35° C. to 65° C., from 40° C. to 60° C. or from 45° C. to 55° C. before performing the spinning cone column distillation on the slurry.

In some embodiments, the slurry is not heated before performing the spinning cone column distillation on the slurry.

In some embodiments, step c) may be performed at a temperature below 110° C. In some embodiments, step c) is performed at a temperature below 110° C., 109° C., 108° C., 107° C., 106° C., 105° C., 104° C., 103° C., 102° C. or 101° C. In some embodiments, step c) is performed at a temperature above 109° C., 108° C., 107° C., 106° C., 105° C., 104° C., 103° C., 102° C., 101° C. or 100° C. In one embodiment, step c) is performed at a temperature between 110° C. and 100° C., between 109° C. and 100° C., between 108° C. and 100° C., between 107° C. and 100° C., between 106° C. and 100° C., between 105° C. and 100° C., between 104° C. and 100° C., between 103° C. and 100° C., between 102° C. and 100° C. or between 101° C. and 100° C.

In some embodiments, the temperature of the slurry during step c) is above room temperature for about 30 to 300 seconds. The temperature of the slurry during step c) may be above room temperature for about 30, 45, 60, 75, 90, 105, 120, 135, 150, 165, 180, 195, 210, 225, 240, 255, 270, 285 or 300 seconds. In one embodiment, the temperature of the slurry during step c) is above room temperature for less than 45, 60, 75, 90, 105, 120, 135, 150, 165, 180, 195, 210, 225, 240, 255, 270, 285 or 300 seconds. In another embodiment, the temperature of the slurry during step c) is above room temperature at least 45, 60, 75, 90, 105, 120, 135, 150, 165, 180, 195, 210, 225, 240, 255, 270, 285 or 300 seconds. In a further embodiment, the temperature of the slurry during step c) is above room temperature for between 30 and 300 seconds, between 30 and 300 seconds, between 30 and 270 seconds, between 30 and 240 seconds, between 30 and 210 seconds, between 30 and 180 seconds, between 30 and 150 seconds, between 30 and 120 seconds, between 30 and 90 seconds or between 30 and 60 seconds.

Without wishing to be bound by any theory, the inventors believe that spinning cone column (SCC) distillation uses steam to separate volatile compounds from liquids or slurries under reduced pressure. This is achieved by causing the garlic slurry to flow as a thin film down a vertical column of spinning and stationary cones within the SCC unit. Simultaneously, steam is introduced into the base of the column, which flows upwards and across the surface of the volatile-rich films of the garlic slurry, thereby separating the volatile compounds from the slurry. This steam then flows through to a condensing system which concentrates the volatile compounds separated from the garlic slurry into a garlic oil extract and a distillate. The condensed garlic oil and distillate can then be collected and bottled separately. The garlic slurry can optionally be heated as it is pumped into the SCC unit in order to increase volatility of the compounds therein. Both the oil and the distillate may contain volatile compounds extracted from the garlic.

Advantageously, the conditions in spinning cone column distillation are very mild. Since SCC is a flow-based method, the garlic slurry is only at an elevated temperature for a relatively short period of time (from 30 seconds up to 1 minute) as it flows through the SCC unit. In comparison, conventional methods must heat the entire garlic mass at once, resulting in extended periods of elevated temperature and therefore increased degradation of the volatile compounds. Additionally, since the interior of the SCC unit is under reduced pressure (and subsequently filled with steam), the environment inside is oxygen-poor. This further reduces the rate at which the bioactive compounds (particularly organosulfates) within the garlic degrade, as any potential for oxidation reactions are minimised.

An additional advantage of this method is that the garlic oil produced has a low water content and is not an emulsion. Emulsions and compositions with a high water content necessarily result in a lower concentration of the active components, and are often not microbially stable. Other methods macerate garlic in oils from other plants (i.e. soybean or vegetable oil) to thereby extract hydrophobic compounds from the garlic and produce a garlic-infused oil. However, these methods necessarily produce a very dilute product, thereby causing a reduction or even complete loss of bioactivity.

In one embodiment, the water content of the garlic oil produced in step c) is less than 5%, less than 4%, less than 3%, less than 2%, less than 1%, less than 0.5% or less than 0.1% v/v.

In some embodiments, step c) is performed at a temperature of below 105° C.

In some embodiments, step c) is performed by supercritical fluid extraction. This technique uses high pressure to generate supercritical solvents (e.g. supercritical CO₂), at a relatively low temperature, which are then used to separate volatile compounds from the garlic. The supercritical fluid extraction may be supercritical carbon dioxide extraction. The supercritical fluid extraction may be supercritical air, ammonia, nitrogen or water extraction. In some embodiments the aged garlic is freeze dried before performing supercritical fluid extraction.

In a fourth aspect of the invention, there is provided a garlic oil when produced by the method of any one of the first to third aspects.

In a fifth aspect of the invention, there is provided a pharmaceutical composition comprising the garlic oil of the fourth aspect. The composition may further comprise a pharmaceutically acceptable carrier, diluent and/or excipient.

While it is possible that the garlic oil of the fourth aspect may be administered as a neat chemical, it also may be administered as part of a pharmaceutical composition which includes at least one carrier or excipient.

The type of pharmaceutical composition may depend upon the Absorption, Distribution, Metabolism and Excretion (ADME) profile of the pharmaceutical composition. The pharmaceutical composition may include those suitable for oral or rectal administration, or for administration by non-intravenous routes. An oral composition for oral administration may be preferred.

Topical administration includes buccal, sub-lingual, dermal, ocular, rectal, nasal, as well as administration by inhalation or by aerosol means. Those of skill in the art would be able to prepare suitable formulations.

The nature of the pharmaceutical composition and the carrier or excipient will depend on the route of administration and the nature of the condition and the patient being treated. It is believed that the choice of a particular carrier, excipient or delivery system, and route of administration could be readily determined by a person skilled in the art. In some circumstances it may be necessary to protect the pharmaceutical composition by means known in the art, for example, by micro encapsulation. The route of administration should also be chosen such that the active agent reaches its site of action. The pharmaceutical composition may include any suitable effective amount of the active agent commensurate with the intended dosage range to be employed.

The pharmaceutical composition may be in the form of a liquid (including solutions, suspensions, syrups, emulsions, colloids, elixirs, creams, gels and foams).

The pharmaceutically acceptable carrier(s) or excipient(s) must be acceptable in the sense of being compatible with the other components in the composition and not being deleterious to the patient. The pharmaceutically acceptable carrier or excipient may be either a solid or a liquid. The carrier or excipient may act as a diluent, buffer, stabiliser, isotonicising agent, flavouring agent, anti-oxidant, solubilizer, lubricant, suspending agent, binder, preservative, tablet disintegrating agent or an encapsulating material. Suitable carriers and excipients would be known to a skilled person. With regard to buffers, aqueous compositions may include buffers for maintaining the composition at close to physiological pH or at least within a range of about pH 6.0 to 9.0.

Liquid form preparations may include, for example, water, saline, water-dextrose, water-propylene glycol, petroleum, or oil (including animal, vegetable mineral or synthetic oil) solutions. For example, parenteral injection liquid preparations may be formulated as solutions in aqueous polyethylene glycol solution. Such liquid form preparations may contain at least 0.1 wt % of the garlic oil.

Liquid pharmaceutical compositions may be formulated in unit dose form and may include a preservative. The compositions may also include formulatory agents such as suspending, stabilising and/or dispersing agents. Liquid carriers and excipients may include colorants, flavours, stabilizers, buffers, artificial and natural sweeteners, dispersants, thickeners, solubilizing agents, suspending agents and the like.

For topical administration to the epidermis the composition may be formulated as an ointment, cream or lotion, or as a transdermal patch.

In a sixth aspect of the invention, there is provided a method of treating or preventing a viral infection, the method comprising administering to a patient in need thereof the garlic oil of the fourth aspect or the pharmaceutical composition of the fifth aspect.

In a seventh aspect of the invention, there is provided a use of the garlic oil of the fourth aspect in the manufacture of a medicament for treating or preventing a viral infection.

In an eighth aspect of the invention, there is provided the garlic oil of the fourth aspect for use in the treatment or prevention of a viral infection.

The viral infection of the sixth to eighth aspects may be selected from at least one of the group consisting of: Adenovirus-3 (AdV-3), Adenovirus-41 (AdV-41), Coronavirus (CoV), Severe acute respiratory syndrome coronavirus (SARS-COV), Dengue virus (DENV), Herpes simplex virus-1 (HSV-1), Herpes simplex virus-2 (HSV-2), Human cytomegalovirus (HCMV), Influenza A virus subtype H9N2 (IAV-H9N2), Influenza B virus (IBV), Influenza A virus-H1N1 (IAV-H1N1), Coxsackie B-3 (CBV-3), Echovirus-11 (ECHO), Enterovirus-71 (EV-71), Human rhinovirus-2 (HRV-2), Hepatitis A virus (HAV), Measles virus (MeV), Newcastle disease virus (NDV), Parainfluenza virus-3 (PIV-3), Vaccinia virus (VV), Vesicular stomatitis virus (VSV), Human immunodeficiency virus-1 (HIV-1), Reticuloendotheliosis virus (REV), Porcine Rotavirus (PRV) and Rotavirus SA-11 (RV-SA-11).

In some embodiments, the viral infection of the sixth to eighth aspects is selected from the group consisting of: SARS-COV-2 and influenza A. In one embodiment, the viral infection of the sixth or seventh aspect is SARS-COV-2 infection. In another embodiment, the viral infection of the sixth or seventh aspect is influenza A.

In a ninth aspect of the invention, there is provided a use of the garlic oil of the fourth aspect in the manufacture of a medicament for treating or preventing a fungal infection.

In a tenth aspect of the invention, there is provided the garlic oil of the fourth aspect for use in the treatment or prevention of a fungal infection.

In an eleventh aspect of the invention, there is provided a pharmaceutical composition comprising a garlic oil.

In a twelfth aspect of the invention, there is provided a method of treating or preventing a viral infection, the method comprising administering to a patient in need thereof garlic oil, fresh garlic or the pharmaceutical composition of the eleventh aspect.

For the twelfth aspect, the viral infection may be selected from the group consisting of: Adenovirus-3 (AdV-3), Adenovirus-41 (AdV-41), Coronavirus (CoV), Severe acute respiratory syndrome coronavirus (SARS-COV), Dengue virus (DENV), Herpes simplex virus-1 (HSV-1), Herpes simplex virus-2 (HSV-2), Human cytomegalovirus (HCMV), Influenza A virus subtype H9N2 (IAV-H9N2), Influenza B virus (IBV), Influenza A virus-H1N1 (IAV-H1N1), Coxsackie B-3 (CBV-3), Echovirus-11 (ECHO), Enterovirus-71 (EV-71), Human rhinovirus-2 (HRV-2), Hepatitis A virus (HAV), Measles virus (MeV), Newcastle disease virus (NDV), Parainfluenza virus-3 (PIV-3), Vaccinia virus (VV), Vesicular stomatitis virus (VSV), Human immunodeficiency virus-1 (HIV-1), Reticuloendotheliosis virus (REV), Porcine Rotavirus (PRV) and Rotavirus SA-11 (RV-SA-11).

In some embodiments, the viral infection of the twelfth aspect is selected from the group consisting of: SARS-COV-2 and influenza A. In some embodiments, the viral infection of the twelfth aspect is SARS-COV-2. In other embodiments viral infection of the thirteenth and fourteenth aspects is influenza A.

In some embodiments of the eleventh and twelfth aspects, the garlic oil is the garlic oil of the fourth aspect.

In a thirteenth aspect of the invention, there is provided a method of treating or preventing a viral infection in a plant, the method comprising administering to the plant an effective amount of the garlic oil of the fourth aspect. In some embodiments, the garlic oil is administered to the leaves, stem, flower, roots or soil around the roots of the plant, or a combination thereof. In one embodiment the viral infection is at least one selected from the group consisting of: Potato Virus Y; Spotted wilt virus, Grapevine leafroll-associated virus 2, Tomato Mosaic Virus and Potyvirus.

In a fourteenth aspect of the invention, there is provided a method of treating or preventing a fungal infection in a plant, the method comprising administering to the plant an effective amount of the garlic oil of the fourth aspect. In some embodiments, the garlic oil is administered to the leaves, stem, flower, roots or soil around the roots of the plant, or a combination thereof. In one embodiment the fungal infection is at least one selected from the group consisting of: Verticillium, Alternaria (such as Alternaria brassicicola), Botrytis cinerea, Magnaporthe (such as Magnaporthe grisea) and Plectospherella (such as Plectospherella cucumerina).

As used herein, the terms “treatment” (or “treating”) and “prevention” (or “preventing”) are to be considered in their broadest contexts. For example, the term “treatment” does not necessarily imply that a patient is treated until full recovery. The term “treatment” includes amelioration of the symptoms of a disease, disorder or condition, or reducing the severity of a disease, disorder or condition. Similarly, “prevention” does not necessarily imply that a subject will never contract a disease, disorder or condition. “Prevention” may be considered as reducing the likelihood of onset of a disease, disorder or condition, or preventing or otherwise reducing the risk of developing a disease, disorder or condition.

As used herein, the terms “subject” or “individual” or “patient” may refer to any subject, particularly a vertebrate subject, and even more particularly a mammalian subject, for whom therapy is desired. Suitable vertebrate animals include, but are not restricted to, primates, avians, livestock animals (e.g., sheep, cows, horses, donkeys, pigs), laboratory test animals (e.g., rabbits, mice, rats, guinea pigs, hamsters), companion animals (e.g., cats, dogs) and captive wild animals (e.g., foxes, deer, dingoes). A preferred subject is a human.

As used herein, “effective amount” refers to the administration of the garlic oil in an amount sufficient to at least partially attain the desired response, or to prevent the occurrence of symptoms of the virus being treated, or to bring about a halt in the worsening of symptoms or to treat and alleviate or at least reduce the severity of the symptoms. The amount may vary depending on factors such as: the health and physical condition of the individual to whom the garlic oil is administered, the taxonomic group of the individual to whom the garlic oil is administered, the extent of treatment/prevention desired, the formulation of the composition, and the assessment of the medical situation. It is expected that the “effective amount” will fall within a broad range that can be determined through routine trials. Dosage regimes may be adjusted to provide the optimum therapeutic response. For example, several doses may be administered daily, bi-weekly or weekly, or at other suitable time intervals, or the dose may be proportionally reduced as indicated by the circumstances. Decisions on dosage and the like would be within the skill of the medical practitioner or veterinarian responsible for the care of the patient.

In the present specification and claims, the word ‘comprising’ and its derivatives including ‘comprises’ and ‘comprise’ include each of the stated integers but does not exclude the inclusion of one or more further integers.

The transitional phrase “consisting of” excludes any element, step, or ingredient not specified. If in the claim, such would close the claim to the inclusion of materials other than those recited except for impurities ordinarily associated therewith. When the phrase “consisting of” appears in a clause of the body of a claim, rather than immediately following the preamble, it limits only the element set forth in that clause; other elements are not excluded from the claim as a whole.

The transitional phrase “consisting essentially of” is used to define a composition, process or method that includes materials, steps, features, components, or elements, in addition to those literally disclosed, provided that these additional materials, steps, features, components, or elements do not materially affect the basic and novel characteristic(s) of the claimed invention. The term “consisting essentially of” occupies a middle ground between “comprising” and “consisting of”.

Also, the indefinite articles “a” and “an” preceding an element or component of the invention are intended to be non-restrictive regarding the number of instances (i.e., occurrences) of the element or component. Therefore “a” or “an” should be read to include one or at least one, and the singular word form of the element or component also includes the plural unless the number is obviously meant to be singular.

As used herein, with reference to numbers in a range of numerals, the terms “about”, “approximately” and “substantially” are understood to refer to the range of −10% to +10% of the referenced number, preferably-5% to +5% of the referenced number, more preferably-1% to +1% of the referenced number, most preferably-0.1% to +0.1% of the referenced number. Moreover, with reference to numerical ranges, these terms should be construed as providing support for a claim directed to any number or subset of numbers in that range. For example, a disclosure of from 1 to 10 should be construed as supporting a range of from 1 to 8, from 3 to 7, from 1 to 9, from 3.6 to 4.6, from 3.5 to 9.9, from 8 to 10, and so forth.

Unless expressly stated to the contrary, “or” refers to an inclusive or and not to an exclusive or. For example, a condition A or B is satisfied by any one of the following: A is true (or present) and B is false (or not present), A is false (or not present) and B is true (or present), and both A and B are true (or present).

Reference throughout this specification to ‘one embodiment’ or ‘an embodiment’ means that a particular feature, structure, or characteristic described in connection with the embodiment is included in at least one embodiment of the present invention. Thus, the appearance of the phrases ‘in one embodiment’ or ‘in an embodiment’ in various places throughout this specification are not necessarily all referring to the same embodiment. Furthermore, the particular features, structures, or characteristics may be combined in any suitable manner in one or more combinations.

Any of the features described herein can be combined in any combination with any one or more of the other features described herein within the scope of the invention.

The reference to any prior art in this specification is not, and should not be taken as an acknowledgement or any form of suggestion that the prior art forms part of the common general knowledge.

### BRIEF DESCRIPTION OF DRAWINGS

Preferred features, embodiments and variations of the invention may be discerned from the following Detailed Description which provides sufficient information for those skilled in the art to perform the invention. The Detailed Description is not to be regarded as limiting the scope of the preceding Summary of the Invention in any way. The Detailed Description will make reference to a number of drawings as follows:

FIG. 1 shows a flowchart of garlic oil production using Spinning Cone Column distillation.

FIG. 2 shows a TCID50 of garlic juice samples, where ** represents p<0.01 compared to inoculum only using one-way ANOVA, Dunnett's multiple comparisons test.

FIG. 3 shows a TCID50 of garlic juice samples compared to controls, where represents p<0.001 compared to inoculum only using one-way ANOVA, Dunnett's multiple comparisons test.

FIG. 4 shows a TCID50 of garlic juice samples 2 and 3 with measures to reduce the detection limit taken, where *** represents p<0.001 compared to the no juice control using one-way ANOVA.

FIG. 5 shows an infectious virus titre assay of oil 1 using ELISA based detection of SARS-COV-2 viral genome, where *** represents p<0.001, two way ANOVA, Dunnett's multiple comparisons test.

FIG. 6 shows an infectious virus titre assay of garlic oils 1 to 5 using ELISA based detection of SARS-COV-2 viral genome where *** represents p<0.001 compared to no treatment control, one way ANOVA, Dunnett's multiple comparisons test.

FIG. 7 shows an infectious virus titre assay of oil 10 using ELISA based detection of SARS-COV-2 viral genome.

FIG. 8 shows an infectious virus titre assay of garlic juice samples against influenza virus (PR8) where *** represents p<0.001 compared to the no treatment control using one-way ANOVA.

FIG. 9 shows an infectious virus titre assay of oil samples against influenza virus (PR8).

### DESCRIPTION OF EMBODIMENTS

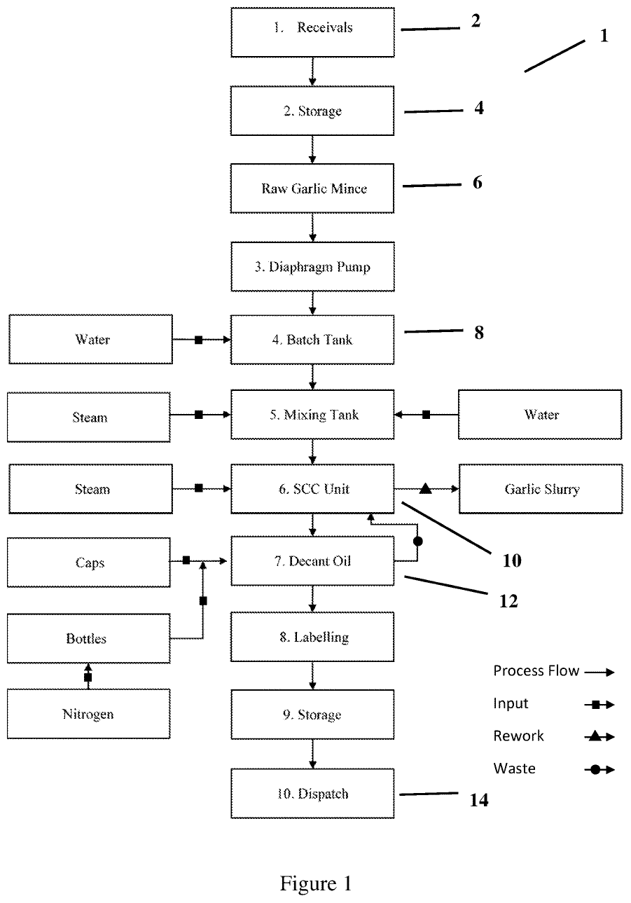

An exemplary method 1 is illustrated in FIG. 1. The method begins with harvesting a garlic plant, for example on a farm. Then the garlic is transported to a facility for further processing 2, and is potentially stored 4. The garlic is then processed to increase its surface area 6, and this may include cutting the top from the garlic and peeling the garlic (although these steps may also be performed prior to step 2), and then mincing the garlic to produce raw garlic mince. The garlic mince is then aged for between 18 months and 5 years, especially at a temperature of around −5° C. to 5° C. The ageing is performed under sterile, food grade conditions.

After ageing, the garlic is pumped to a batch tank 8, where water is added to provide a slurry of an appropriate consistency. The slurry may be preheated (for example to about 50-55° C.) prior to use in the spinning cone column 10. The garlic oil is then decanted 12, before labelling, storage and dispatch 14.

### Example 1: Garlic oil preparation

### Processing of Raw Garlic

To prepare garlic oil, garlic heads were first harvested and separated into cloves. The cloves were then peeled to remove the skin and minced in order to produce a raw garlic mince. The raw garlic mince was aged in bins at a temperature between −3° C. and 3° C. for between 18 months and 5 years before use in production of garlic oil. The garlic mince was aged in sterile, food grade conditions so as to avoid production of harmful microorganisms.

The garlic varieties used to produce the garlic oil and time that each were aged for are outlined in Table 1 below.

TABLE 1
Garlic varieties and age of garlic mince used to prepare oil extracts.

| Garlic | | | Age of garlic |
|---|---|---|---|
| varieties | Variety name | Botanical Name | mince |
| MOF | Australian RED | Allium Sativum | 3 Years |
| WHT | Australian WHITE | Allium Sativum | 4 Years |
| AUP | Australian PURPLE | Allium Sativum | 5 Years |

Production of Garlic Oil from Aged Garlic Mince

Garlic oil was extracted from aged garlic mince through spinning cone column (SCC) distillation. To extract garlic oil using SCC distillation, water was added to the raw garlic mince to provide a garlic slurry. The amount of water added was adjusted as necessary to provide an appropriate consistency. The garlic slurry was then preheated to about 50° C.-55° C. before being pumped into the SCC unit. The SCC unit was operated using the following parameters:

TABLE 2
Machine parameters for SCC unit

| Product flow rate | 450 | L/Hr |
|---|---|---|
| Product heater setpoint mode | Local SP | |
| Product heater temperature - Local SP | 98.0 | °C |
| Product heater offset - Remote SP | −2.0 | °C |

| | |
|---|---|
| SCC internal strip rate | 8.2% |
| Condenser 1 Strip Rate | 0.0% |
| SCC discharge pump 1 speed | 40.0% |
| SCC discharge pump 2 speed | 40.0% |

The product flow rate may be varied but was preferably between about 425 L/hr and 475L/Hr. The product heater temperature was between about 96° C. and about 100° C. Preferably, the product heater temperature was greater than about 95° C. and less than about 101° C. The SCC top vapour temperature was between about 98° C. to 101° C. The SCC bottom product temperature was between about 98° C. to 101° C. The top vapour and bottom product temperatures were preferably similar. The difference between the top vapour and bottom product temperatures was generally less than about 1.5° C. to 2° C. The steam valve position was preferably less than 100%.

### Chemical Analysis of the Oils

Using the above method, oil samples 1-4 were prepared from different of garlic varieties, as set out in Table 3. Sample 5 was sourced from commercially available garlic oil produced from unknown second grade (mixed) Spanish garlic variety(ies) that was prepared by traditional distillation methods. Sample 6 was pure olive oil and represents a control that did not include any garlic oil.

TABLE 3
Oils by variety

| Sample Number | Sample ID | Garlic Varieties present |
|---|---|---|
| O 1 | SupaG MOFO | MOF |
| O 2 | SupaG WHTO | WHT |
| O 3 | SupaG AUPO | AUP |
| O 4 | SupaG M/W/AO | MOF/WHT/AUP blend |
| O 5 | SupaG AUPSO | Spanish Garlic |
| O 6 | BBOO | N/A |

Each of the above oils were analysed by Gas Chromatography Mass Spectrometry (GCMS) using an Agilent 6890 Gas Chromatograph coupled to an Agilent 5973 Mass Spectrometer, the results of which are detailed in Table 4.

TABLE 4
Relative abundance (%) of major components of the garlic oils

| | | methyl | cis | trans | | | | | methyl | | di | di | di |
|---|---|---|---|---|---|---|---|---|---|---|---|---|---|
| | | allyl | propenyl | propenyl | dimethyl | allyl | Allyl-1- | Allyl-1- | allyl | 2-vinyl- | allyl | (1- | allyl |
| Sample | allyl | di- | methyl | methyl | tri- | di- | prepenyl | prepenyl | tri- | 4h-1,3- | tri- | propenyl) | tetra |
| Number | sulfide | sulfide | disulfide | disulfide | sulfide | sulfide | isomer 1 | isomer 2 | sulfide | dithiin | sulfide | sulfide | sulphide |
| O 1 | 2.5 | 2.2 | 0.4 | 1.9 | 1.0 | 15.6 | 3.2 | 13.1 | 15.1 | 1.2 | 32.5 | 0.9 | 0.9 |
| O 2 | 3.5 | 3.0 | 0.4 | 1.1 | 0.9 | 17.5 | 3.2 | 8.5 | 14.5 | 1.1 | 34.2 | 1.5 | 0.3 |
| O 3 | 2.7 | 2.4 | 0.4 | 1.4 | 0.9 | 16.4 | 3.3 | 10.6 | 15.0 | 1.2 | 35.0 | 1.1 | 0.2 |

As seen in Table 5 below, the organosulfur compounds present in the oils make up a high percentage of the isolated compounds. This data shows that the above described method is able to substantially preserve the high levels of organosulfur compounds found in raw garlic.

TABLE 5
Relative abundance of sulfur containing compounds in oils 1-3

| Sample Number | Total Sulfur Compounds (%) |
|---|---|
| O 1 | 90.50 |
| O 2 | 89.70 |
| O 3 | 90.60 |

### Example 2: Garlic Juice Preparation

To prepare garlic juice, freshly harvested garlic bulbs were cracked and peeled to produce peeled garlic cloves. The peeled garlic cloves were then juiced using a domestic juicer to produce a mixture of garlic juice and garlic pulp. The garlic pulp was then filtered using filter paper to separate the garlic juice. Using this method, garlic juice was prepared from a range of garlic varieties according to Table 6.

TABLE 6
Garlic Juice samples by variety

| Sample Number | Variety |
|---|---|
| GJ 1 | AUP |
| GJ 2 | WHT |
| GJ 3 | WHT |

### Example 3: Antiviral Activity of Garlic Juice Against SARS-COV-2 Via Infectious Titre Assay

Garlic Juices 2 and 3 were then tested for viricidal activity at the highest non-cytotoxic concentration against SARS-COV-2 (human isolate-delta variant, isolate Victoria/18440/2021) via infectious titre assay.

### Method

To perform the experiment, known amounts of SARS-COV-2 were added to fresh garlic juice and incubated at room temperature for 30 min. Following this, the change in infectious titre was determined by performing a TCID50. The experiment was repeated to ensure the scientific integrity of the findings.

First, 96 well plates were seeded with Vero cells and cultured at 37° C. under 5% CO₂ overnight. Then 900 μL of each Garlic Juice sample was added to individual Eppendorf tubes. 100 μL of SARS-COV-2 stocks (delta variant (isolate Victoria/18440/2021)) was then added to each sample and sealed. Samples were shaken by hand every 5 min to ensure as much mixing as possible. At 30 min, samples were serially diluted (⅕s) and a TCID50 performed to determine if there had been any change in infectious virus titre. Supernatants taken from the initial TCID50 were passaged a second time to determine presence of any remaining infectious virus.

### Results

It was not possible to discern microscopically whether there was virus induced cytopathic effect (CPE) in the undiluted, 5⁻¹ and 5⁻² wells. Samples were taken from these wells, as well as the 5⁻³, 5⁻⁴ and 5⁻⁵ and added to new Vero cell monolayers to assess for presence of infectious virus.

Cells in the undiluted, 5⁻¹ and 5⁻² diluted wells were dead due to an obliterated monolayer. Therefore, the limit of detection (LOD) of this assay was 103.5 TCID50/mL. Virus induced CPE was recorded when detected via microscopic examination of each well. It was found that the majority of the samples appeared to have a higher incidence of CPE presence in the wells than the inoculum only. This could be due to either direct effects of the garlic juice, or intensity of the odour affecting the cultures.

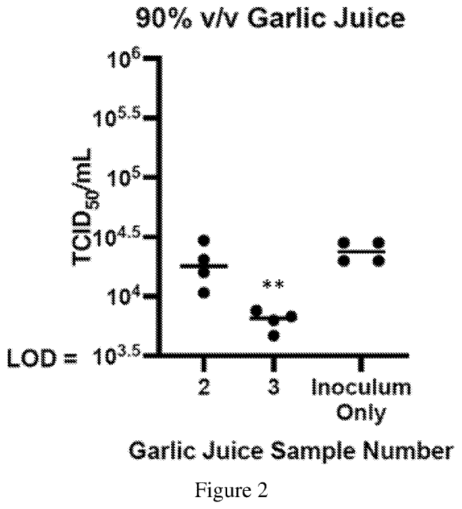

As best seen in FIG. 2 and Table 7, garlic juices 2 and 3 significantly reduced the infectious titre compared to the inoculum only control. Given the limit of detection, this resulted in up to a 55% and 80.67% reduction in infectious titre respectively compared to the inoculum only control.

TABLE 7
Percentage (%) Reduction in infectious virus titre

Sample

| Replicate | GJ 2 | GJ 3 |
|---|---|---|
| ¹ | 14.17 | 73.71. |
| 2 | 34.17 | 80.67 |
| 3 | 55.00 | 68.75 |
| 4 | Not reduced | 71.63 |

### Example 4: Antiviral Activity of Garlic Juice Against SARS-COV-2 Via Infectious Titre Assay

The above assay was repeated using uninfected controls containing the same dilutions of garlic juice that would be achieved in samples generated from exposing the juice to the virus. This experiment investigated whether the cytotoxicity was contributed to by the garlic juice or presence of infectious virus, thereby enabling conclusive determination of the effect of the juices on the virus infectious titre.

### Method

First, 96 well plates were seeded with Vero cells and cultured at 37° C. under 5% CO₂ overnight. Then 450 uL of each garlic juice sample was added to individual Eppendorf tubes in duplicate and transferred to the PC3 laboratory. 50 uL of SARS-COV-2 stocks (delta variant (isolate Victoria/18440/2021)) was then added to a single tube of each sample and sealed. The second tube had only media added (no virus). Samples were shaken by hand every 5 min to ensure as much mixing as possible. At 30 min, samples were serially diluted (⅕s) and a TCID50 performed to determine if there had been any change in infectious virus titre. In case the noxious odour was affecting wells when samples were plated undiluted, the TCID50 started at the 5⁻¹ dilution. Supernatants taken from the initial TCID50 were passaged a second time to determine presence of any remaining infectious virus.

### Results

For both uninfected sample controls, and samples that had been exposed to virus, the 5⁻¹ and 5⁻² wells were impossible to discern microscopically whether there was virus induced CPE due to presence of garlic juice. At the 5⁻³ dilution, significant cell death was noted in the wells containing the uninfected sample controls. Therefore, beginning at the 5⁻³ dilution, a second passage of samples was taken from both the infected and uninfected control wells to determine whether there was continued cell death from the presence of juice, or whether virus induced CPE could be discerned.

For the second passage of samples from the uninfected control wells, all monolayers appeared healthy and no cell death was noted. Virus induced CPE was recorded when detected via microscopic examination of each well.

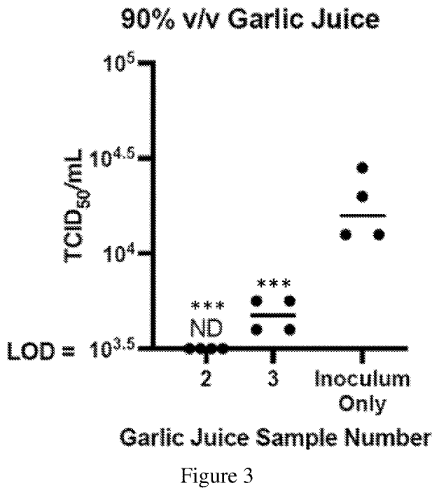

As best seen in FIG. 3, and Table 8, garlic juices 2 and 3 reduced the infectious titre of SARS-COV-2 to non-detectable (ND) levels. Given the limit of detection, this equated to an 82.7% and 73.8% reduction in infectious titre respectively compared to the inoculum only control. A one-way ANOVA (Dunnett's multiple comparisons test) of Garlic Juices 2 and 3 showed a significance of p<0.001 compared to inoculum only. Surprisingly, Garlic juice 2 produced a TCID50 that was below the limit of detection for this assay.

TABLE 8
Percentage (%) Reduction in infectious virus titre.

Sample

| Replicate | GJ 2 | GJ 3 |
|---|---|---|
| 1 | 82.73 | 69.29 |
| 2 | 82.73 | 78.25 |
| 3 | 82.73 | 78.25 |
| 4 | 82.73 | 69.29 |
| Average | 82.73 | 73.8 |

### Example 5: Antiviral Activity of Garlic Juice Against SARS-COV-2 Via Infectious Titre Assay

As the experiment of Example 4 surprisingly produced a TCID50 value below the detection limit of the assay, a repeat experiment was performed, with modifications made in order to lower the detection limit. This experiment used garlic juice numbers 2 and 3 as well as a control without garlic juice.

### Method

The method was as outlined under Example 4, with the following modifications: (a) At the time of sampling, 200 μL of eluate was taken from samples exposed to 2 and 3, as well as the no juice control and added directly into the RNA extraction buffer; and (b) Samples of supernatant were also taken and passaged a second time in Vero cells and thus reduced the detection limit of the assay.

### Results

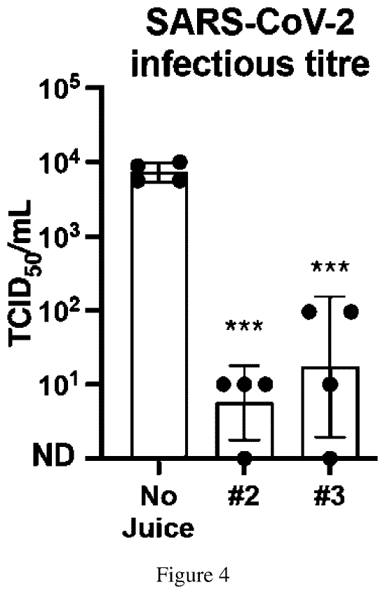

As best seen in Table 9 and FIG. 4, garlic juice 2 and garlic juice 3 reduced the infectious titre of SARS-COV-2 to nearly non-detectable (ND) levels, which equated to an average of 99.9% and 99.3% (respectively) reduction in infectious titre compared to the no juice control.

TABLE 9
Percentage (%) Reduction in infectious virus titre

Sample

| Replicate | GJ 2 | GJ 3 |
|---|---|---|
| 1 | 99.87 | 99.87 |
| 2 | 99.87 | 99.99 |
| 3 | 99.99 | 98.74 |
| 4 | 99.87 | 98.74 |
| Average | 99.90 | 99.33 |

### Example 6: Antiviral Activity of Garlic Oil Against SARS-COV-2

Garlic oil 1 was tested against SARS-COV-2 human isolate-delta variant (isolate Victoria/18440/2021) via infectious titre assay.

### Method

The method was as follows:
1. Oils were heated in a water bath (37° C.) until completely in solution, then allowed to cool to room temperature.
2. Oil test solutions were made by:
   a. Creating a 1/50 (100 μL oil plus 4.9 mL infection media) dilution
   b. Creating a 1/500 (0.5 mL of 1/50 dilution plus 4.5 mL infection media) dilution
3. Samples were vortexed for 30 sec then added to either DMSO or isopropanol as follows:
   a. DMSO=10 uL DMSO+990 uL of either of the diluted oil samples; final concentration of DMSO=1% v/v
   b. Isopropanol=100 uL isopropanol+900 uL of either of the diluted oil samples; final concentration of isopropanol=10% v/v
4. Samples were vortexed for at least 30 sec and monitored for separation. Solutions appeared cloudy/emulsified and stable. Two samples for each test condition were created.
5. 100 uL of SARS-COV-2 stocks (delta variant (isolate Victoria/18440/2021)) was added to test solutions and sealed.
6. Samples were shaken by hand every 5 min to ensure as much mixing as possible. The solutions appeared cloudy/emulsified throughout the incubation period (30 min at room temperature).
7. At 30 min, samples were serially diluted and a TCID50 performed to determine if there had been any change in infectious virus titre.
8. In order to detect the presence of infectious virus, an Enzyme-Linked Immunosorbent Assay (ELISA) was used.

### Results

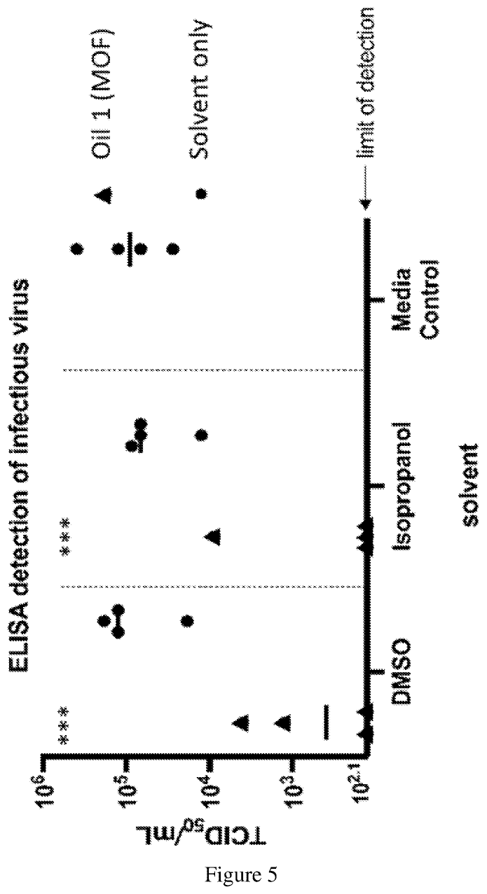

As best seen in Table 10 and FIG. 5, solutions prepared using garlic oil 1 (MOF) exhibited significant virucidal activity against SARS-COV-2 when solubilized at 1/50 dilution in both DMSO and isopropanol (*** p<0.001, 2 way ANOVA, Dunnett's multiple comparisons test). When solubilized in DMSO, garlic oil 1 reduced the infectious SARS-COV-2 titre by an average of at least 98.6% and when solubilized in isopropanol the infectious titre was reduced by an average of at least 97.71%. As DMSO and isopropanol control samples did not yield any virucidal activity against SARS-COV-2 (the titres were not different to the no solvent control), these results are due to the viricidal effects garlic oil itself.

TABLE 10
Percentage (%) reduction in infectious titre of SARS-CoV-2
human isolate - delta variant after exposure to garlic
oil 1 (O1) solubilised in either DMSO or isopropanol.

| | % Reduction in viral titre | % Reduction in viral titre |
|---|---|---|
| Replicate | (DMSO) | (Isopropanol) |
| 1 | 99.87 | 99.87 |
| 2 | 99.87 | 91.24 |
| 3 | 95.93 | 99.87 |
| 4 | 98.72 | 99.87 |
| Average | 98.6 | 97.7 |

Having confirmed that garlic oil 1 can cause significant reduction in infectious titre, oils 1-3, 5 and 6 were analysed for viricidal activity against SARS-COV-2 using the above method. Solubilization was not required to obtain TCID50 values for Oil 6.

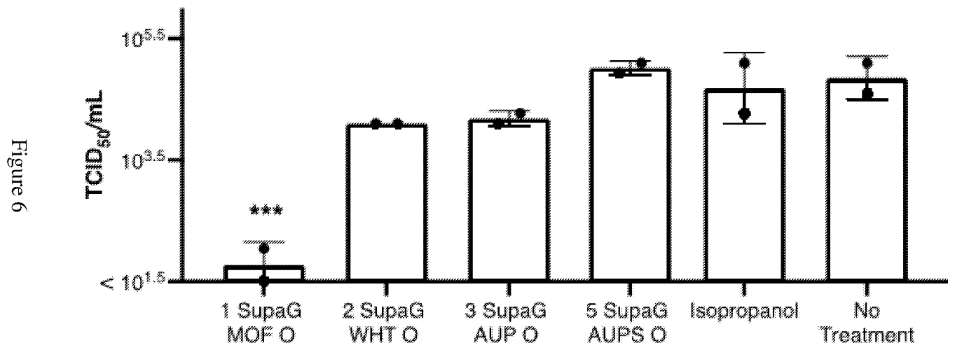

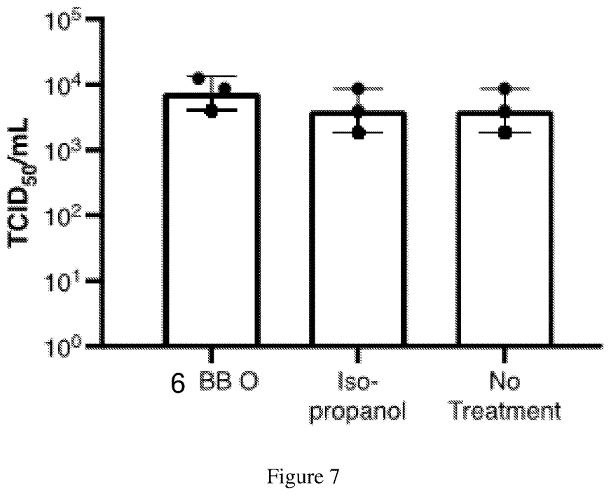

As best seen in Table 11 and FIGS. 6 and 7, garlic oils 1-3 showed significant reduction in viral titre (99.90%, 82.29% and 78.07% respectively). In particular, garlic oil 1 showed significant reduction in viral titre (p<0.001, one-way ANOVA, Dunnett's multiple comparisons test). In contrast, samples 5 and 6 did not result in any reduction in viral titre.

TABLE 11
Percentage (%) reduction in infectious titre

| Sample number | % reduction in viral titre |
|---|---|
| O 1 | 99.90 |
| O 2 | 82.29 |
| O 3 | 78.07 |
| O 5 | Not reduced |
| O 6 | Not reduced |
| Isopropanol control | Not reduced |

### Example 7: Garlic Juice Versus Influenza a Virus Via Infectious Titre Assay

This experiment shows the viricidal activity of fresh garlic juice against influenza A (A/Puerto Rico/8/1934; PR8) via infections titre assay.

### Method

First, 96 well plates were seeded with Vero cells and cultured at 37° C., 5% CO₂ overnight. Then 450 uL of each Garlic Juice sample was added to individual Eppendorf tubes in duplicate in the PC2 laboratory. 50 uL of influenza A virus stocks (isolate A/Puerto Rico/8/1934; also known as PR8) was then added to each sample and sealed. For the second aliquot, 50 uL of infection media was added (uninfected control). Samples were shaken by hand every 5 min to ensure as much mixing as possible. At 30 min, samples were serially diluted (⅕s) and a TCID50 performed to determine if there had been any change in infectious virus titre. 3 days later, supernatant was added to chicken red blood cells to perform a Hemagglutination (HA) titre assay. For wells that contained infectious virus, the red blood cells agglutinate, while the samples that do not contain infectious virus, the red blood cells form a pellet. This gives a clear and definitive answer as to presence of infectious virus.

It was known from previous experiments that the undiluted Garlic Juice would have been toxic to the cells, so the TCID50 was started at ⅕ dilution. Therefore, the limit of detection of infectious virus was 101.85 TCID50/mL. For all wells inoculated with serially diluted Garlic Juice that was not exposed to virus, the Hemagglutination assay revealed no agglutination, therefore Garlic Juice did not interfere with the ability to detect infectious influenza virus using this method.

### Results

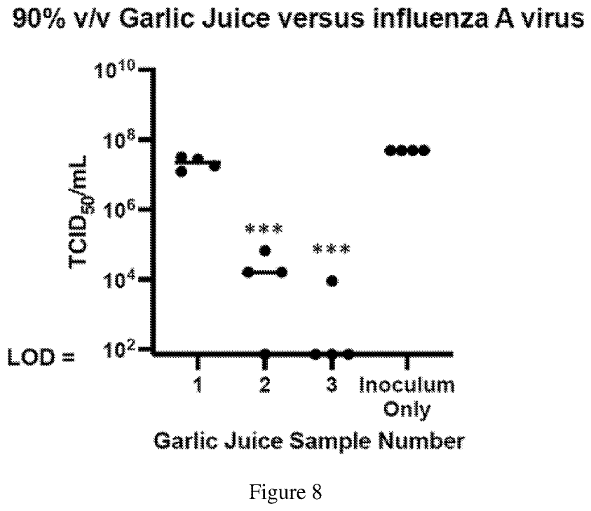

As best seen in FIG. 8 and Table 12, garlic juices 1, 2 and 3 caused a 54.91%, 99.95% and 100% average reduction in the infectious titre of influenza A.

TABLE 12
Percentage (%) Reduction in infectious virus
titre of Influenza A by garlic juice

| | Garlic juice sample | | |
|---|---|---|---|
| Replicate | 1 | 2 | 3 |
| 1 | 36.64 | 99.97 | 99.98 |
| 2 | 43.72 | 99.87 | 100.00 |
| 3 | 64.37 | 100.00 | 100.00 |
| 4 | 74.90 | 99.97 | 100.00 |
| Average | 54.91 | 99.95 | 100.00 |

### Example 8: Antiviral Activity of Garlic Oils Against Influenza A Virus

To determine the viricidal activity of garlic oils, a TCID50 assay was used to measure the infectious titre of influenza A virus (A/Puerto Rico/8/1934; PR8). Confirmation of the presence of virus in samples after microscopic examination was achieved by performing a hemagglutination assay using chicken red blood cells.

### Method

Oils were heated in a water bath (37° C.) until completely in solution, then allowed to cool to room temperature. Test solutions were made by first creating a 1/50 (100 uL oil plus 4.9 mL in saline) dilution. The diluted samples were then vortexed for 30 sec and added to isopropanol in a ratio of 100 uL isopropanol to 900 uL for each of the garlic oil samples (final concentration of isopropanol=10% v/v). Samples were vortexed for at least 30 sec and monitored for separation. Solutions appeared cloudy/emulsified and stable. 100 uL of PR8 stocks was added to test solutions and sealed. Samples were vortexed every 5 min to ensure as much mixing as possible. The solutions remained cloudy/emulsified throughout the incubation period (30 min at room temperature). At 30 min, samples were serially diluted and a TCID50 performed to determine if there had been any change in infectious virus titre. An HA assay was performed on TCID50 supernatants to confirm presence of infectious virus in each well. Given results of the hemagglutination assay were clear, there was no need for a second passage of the TCID50 to confirm presence of infectious virus (or not).

### Results

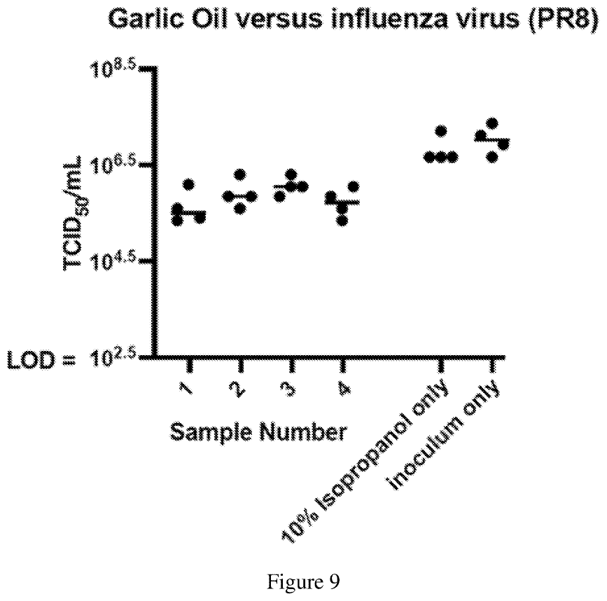

As best seen in table 13 and FIG. 9, an approximate 1-log reduction in influenza infectious titre was observed when the virus was exposed to garlic oil samples 1-4. The cell monolayer appeared intact and similar to the uninfected control wells, indicating the oils did not interfere with MDCK cell viability at the dilutions tested.

TABLE 13
Percentage (%) Reduction in infectious virus titre.

Sample

| Replicate | 1 | 2 | 3 | 4 | 10% isoprop. |
|---|---|---|---|---|---|
| 1 | 98.49 | 97.31 | 95.76 | 97.31 | 62.25 |
| 2 | 99.04 | 97.31 | 95.76 | 95.76 | 62.25 |
| 3 | 99.15 | 98.49 | 97.31 | 98.49 | 62.25 |
| 4 | 95.22 | 92.43 | 92.43 | 99.15 | −28.77 |
| Average | 97.98 | 96.39 | 95.32 | 97.68 | 39.50 |

In compliance with the statute, the invention has been described in language more or less specific to structural or methodical features. It is to be understood that the invention is not limited to specific features shown or described since the means herein described comprises preferred forms of putting the invention into effect. The invention is, therefore, claimed in any of its forms or modifications within the proper scope of the appended claims appropriately interpreted by those skilled in the art.

1. A method of preparing a garlic oil, the method comprising the steps of:
a) processing a portion of a garlic plant to increase its surface area;
b) ageing the portion of the garlic plant for at least 18 months to provide an aged garlic; and
c) separating the garlic oil in the aged garlic, wherein the separating is performed using spinning cone column distillation or supercritical fluid extraction, or wherein the separating is performed at a temperature below 110° C., or wherein during the separating the pressure (atmospheres) divided by the temperature (degrees Kelvin) is equal to or less than about 0.0026,
wherein step b) does not comprise addition of ethanol.

2. The method of claim 1, wherein in step c) the separating is performed at a temperature below 110° C.

3. The method of claim 1, wherein in step c) during the separating the pressure (atmospheres) divided by the temperature (degrees Kelvin) is equal to or less than about 0.0026.

4. The method of claim 1, wherein step a) comprises cutting, grinding, crushing or any combination thereof, or wherein step a) comprises mincing.

5. (canceled)

6. The method of claim 1, wherein step b) does not comprise addition of solvents.

7. The method of claim 1, wherein step b) is performed at a temperature from about −3° C. to about 3° C.

8. The method of claim 1, wherein step b) does not comprise freezing.

9. The method of claim 1, wherein step b) is performed for about 3 to about 5 years.

10. The method of claim 1, wherein step b) is performed at atmospheric pressure.

11. The method of claim 1, wherein in step a) the portion of a garlic plant is peeled garlic cloves.

12. The method of claim 1, wherein step c) is performed using spinning cone column distillation.

13. The method of claim 12, wherein step c) comprises adding an aqueous solution to the aged garlic to form a slurry before performing the spinning cone column distillation on the slurry.

14. The method of claim 13, wherein the slurry is heated to about 50° C. before performing the spinning cone column distillation on the slurry.

15. (canceled)

16. The method of claim 1, wherein the supercritical fluid extraction is supercritical carbon dioxide extraction.

17. The method of claim 1, wherein the portion of a garlic plant is obtained from a garlic variety selected from the group consisting of: Australian White, Australian Red and Australian Purple.

18. The method of claim 1, wherein the portion of a garlic plant is not obtained from a Griffith White garlic variety.

19. A garlic oil when produced by the method of claim 1.

20. (canceled)

21. A method of treating or preventing a viral infection, the method comprising administering to a patient in need thereof the garlic oil of claim 19.

22. The method of claim 21, wherein the viral infection is selected from the group consisting of: SARS-COV-2 and influenza A.

23. (canceled)

24. (canceled)

25. The method of claim 1, wherein in step c) the separating is performed using spinning cone column distillation or supercritical fluid extraction.
